(19) United States
(12) Patent Application Publication
(10) Pub. No.: US 2012/0150044 A1
(43) Pub. Date: Jun. 14, 2012

(54) PHOTOTHERAPY DEVICE AND METHOD

(75) Inventor: Jin Tae KIM, Daejeon (KR)

(73) Assignee: ELECTRONICS AND TELECOMMUNICATIONS RESEARCH INSTITUTE, Daejeon (KR)

(21) Appl. No.: 13/303,181

(22) Filed: Nov. 23, 2011

(30) Foreign Application Priority Data
Dec. 14, 2010 (KR) 10-2010-0127543

(51) Int. Cl. A61B 6/00; A61N 5/06

(52) U.S. Cl. 600/476; 607/90

(57) ABSTRACT

Disclosed is a phototherapy device including: a pixel unit to irradiate light having a predetermined wavelength to a skin, detect a skin reflection light of the light irradiated to the skin, and convert the detected skin reflection light into an electrical signal; an image processor to use the electrical signals to generate skin image information; and a control unit to control light intensity irradiated to the skin for a skin diagnosis or a skin disease therapy based on the skin image information, or to determine a two-dimensional distribution of the light. The phototherapy device of the present disclosure simultaneously provides the diagnosis and therapy of the skin disease.

### CROSS-REFERENCE TO RELATED APPLICATIONS

This application is based on and claims priority from Korean Patent Application No. 10-2010-0127543, filed on Dec. 14, 2010, with the Korean Intellectual Property Office, the disclosure of which is incorporated herein in its entirety by reference.

### TECHNICAL FIELD

The present disclosure relates to a light source-based phototherapy device and method, and more particularly, to a phototherapy device and method capable of simultaneously providing diagnosis and therapy functions of a skin disease.

### BACKGROUND

Recently, a therapy technology of a skin disease such as phototherapy using light sources, such as a laser or a light emitting diode, has been in the limelight.

In particular, the skin disease therapy through the phototherapy device using the light emitting diode may selectively irradiate a specific wavelength light having a safe light intensity to a wide area of exposed skin.

However, the conventional phototherapy device using the light emitting diode provides only a treating function of a skin disease, but does not provide a diagnosing function of a skin disease.

### SUMMARY

The present disclosure has been made in an effort to provide a phototherapy device and method capable of simultaneously performing a diagnosis and therapy of a skin disease.

An exemplary embodiment of the present disclosure provides a phototherapy device, including: a pixel unit to irradiate light having a predetermined wavelength to a skin, detect skin reflection light of the light irradiated to the skin, and convert the detected skin reflection light into an electrical signal; an image processor to use the electrical signal to generate skin image information; and a control unit to control the light intensity irradiated to the skin, or to determine two-dimensional distribution of light for a skin diagnosis or a skin disease therapy based on the skin image information.

Another exemplary embodiment of the present disclosure provides a pixel module for phototherapy, including a plurality of pixels, the pixel module comprising: a first light source to irradiate a first wavelength light in order to acquire skin image information; a second light source to irradiate a second wavelength light for skin diagnosis; a third light source to irradiate a third wavelength light for skin disease therapy; and a photodetector to detect light irradiated to and reflected from the skin, and convert the detected light into electrical signal, the light being irradiated from the first light source or the second light source.

Yet another exemplary embodiment of the present disclosure provides a phototherapy method, including: irradiating a first wavelength light to a skin; generating first skin image information from a reflection light of the first wavelength light reflected from the skin; controlling the intensity of a second wavelength light for a skin diagnosis based on the first skin image information; irradiating the second wavelength light having the controlled light intensity to the skin; generating second skin image information from a reflection light of the second wavelength light reflected from the skin; performing the skin diagnosis based on the second skin image information and determining two-dimensional distribution of a third wavelength light for a skin disease therapy according to the skin diagnosis results; and irradiating the third wavelength light having the determined two-dimensional distribution to the skin.

The phototherapy device and method according to the exemplary embodiments of the present disclosure may control the intensity of the light irradiated for a skin diagnosis based on the skin image information acquired through the skin reflection light of the light irradiated on the skin in order to acquire the skin image information. Furthermore, the phototherapy device and method according to the exemplary embodiments of the present disclosure controls the light intensity to treat the skin disease based on the skin image information acquired through the skin reflection light of the light irradiated for the skin diagnosis, and irradiates the light having the controlled light intensity to the skin, such that the diagnosis and therapy of the skin disease may be simultaneously provided.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a block diagram of the configuration of a phototherapy device, according to an exemplary embodiment of the present disclosure.

FIG. 2 is a diagram showing in detail the configuration of a pixel unit as shown in FIG. 1.

FIGS. 3A to 3C are exemplified diagrams for explaining an operational state of the phototherapy device, according to an exemplary embodiment of the present disclosure.

FIG. 4 is a flow chart of a phototherapy method according to another exemplary embodiment of the present disclosure.

### DETAILED DESCRIPTION

In the following detailed description, reference is made to the accompanying drawing, which form a part hereof. The illustrative embodiments described in the detailed description, drawing, and claims are not meant to be limiting. Other embodiments may be utilized, and other changes may be made, without departing from the spirit or scope of the subject matter presented here.

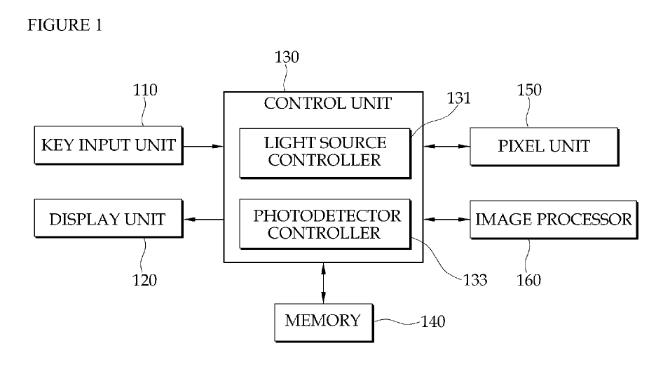

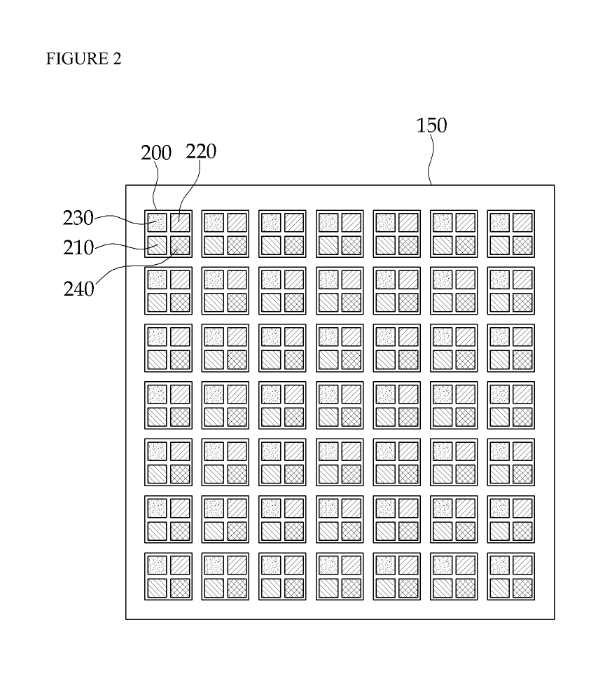

FIG. 1 is a block diagram of the configuration of a phototherapy device according to an exemplary embodiment of the present disclosure, and FIG. 2 is a diagram showing in detail a configuration of a pixel unit as shown in FIG. 1.

Referring to FIGS. 1 and 2, the phototherapy device according to the exemplary embodiment of the present disclosure is configured to include a key input unit 110, a display unit 120, a control unit 130, a memory 140, a pixel unit 150, and an image processor 160.

Key input unit 110 may include an alphanumeric key and various function keys, and outputs key input signals corresponding to a key input by a user to controller 130. The user uses a key included in key input unit 110 to send instructions for a skin diagnosis and therapy through the key input.

Display unit 120 may be configured to include a liquid crystal display (LCD) and displays various display data generated from the phototherapy device according to the exemplary embodiment of the present disclosure. When the LCD is implemented in a touch screen manner, display unit 120 and key input unit 110 may be integrally configured.

Control unit 130 controls overall operations of the phototherapy device according to the exemplary embodiment of the present disclosure. Controller 130 may be configured to include a light source controller 131 to control the light intensity by controlling the light source of pixel unit 150, and a photodetector controller 133 for controlling a photodetector 240 that converts optical signals into electrical signals.

Control unit 130 controls the intensity of the light irradiated from the light source included in pixel unit 150 for a skin diagnosis or therapy of a skin disease, based on skin image information acquired through skin reflection light of the light irradiated to a skin.

That is, control unit 130 irradiates a first wavelength light to a skin through pixel unit 150, and when first skin image information on the skin reflection light of the first wavelength light irradiated to the skin is generated, controls the intensity of a second wavelength light for a skin diagnosis, based on the generated first skin image information.

Control unit 130 controls pixel unit 150 so that the second wavelength light having the controlled light intensity is irradiated to the skin.

When second skin image information on the skin reflection light of the second wavelength light irradiated to the skin through pixel unit 150 is generated, control unit 130 derives skin diagnosis results based on the generated second skin image information.

Control unit 130 analyzes information on a skin-related structure including a skin surface state, the thickness of a skin dermis, the density of a skin dermis, and the position and the shape of a skin disease from the second skin image information to perform the skin diagnosis.

Control unit 130 determines two-dimensional distribution of a third wavelength light to treat a skin disease based on the derived skin diagnosis results, and controls pixel unit 150 to irradiate the third wavelength light to the skin according to the determined two-dimensional distribution.

Control unit 130 may store the acquired skin image information, the skin diagnosis results, and the skin disease therapy results in memory 140 and provide them to a user whenever the need arises.

Meanwhile, when the pixels included in pixel unit 150 are in plural, control unit 130 can control the intensity of the second wavelength light or the third wavelength light for each pixel.

The reason is that all the light intensity for diagnosis for each skin position and all the light intensity to treat a disease of each skin part according to the skin diagnosis results cannot be the same.

Image processor 160 receives electrical signals for the skin reflection light from photodetector 240 to generate two-dimensional or three-dimensional skin image information, and transmits the generated skin image information to control unit 130.

Memory 140 stores various information required to control the operation of the phototherapy device, according to the exemplary embodiment of the present disclosure. In addition, memory 140 may store the skin image information for the skin diagnosis and the skin diagnosis results analyzed from the skin image information.

Pixel unit 150 includes at least one pixel and irradiates light having a predetermined wavelength (for example, from about 400 nm to about 900 nm) to the skin and detects the skin reflection light of the light irradiated to the skin, and converts the detected skin reflection light into the electrical signals.

The configuration of pixel unit 150 will be described with reference to FIG. 2.

Pixel unit 150 has a plurality of pixels 200 two-dimensionally arranged. Pixel 200 is configured to include a first light source 210 to irradiate the first wavelength light in order to acquire the skin image information, a second light source 220 to irradiate the second wavelength light for skin diagnosis, a third light source 230 to irradiate the third wavelength light for the skin disease therapy, and photodetector 240 to detect the skin reflection light of the light irradiated from first light source 210 or second light source 220.

First light source 210, second light source 220, and third light source 230 may be implemented as a light emitting diode (LED) and photodetector 240 may be implemented as a photodiode (PD).

Variables including an output density, a spot size, an operation time, a radiation repetition time, and a radiation repetition period in the light source included in each pixel of pixel unit 150 may be independently controlled by light source controller 131.

The intensity of the second wavelength light or the third wavelength light may be the same or different for each pixel 200. That is, when ten (10) pixels 200 are arranged, the intensity of the second wavelength light or the third wavelength light irradiated from the ten (10) pixels 200 may be different from each other.

FIG. 2 separately shows first light source 210, second light source 220, and third light source 230. However, three light sources may be implemented as one or two light sources when the wavelength of light can be controlled.

Photodetector 240 converts light irradiated from first light source 210 or second light source 220 into electrical signals and transfers the electrical signals to photodetector controller 133. Photodetector controller 133 processes the electrical signals and transfers the processed electrical signals to image processor 160.

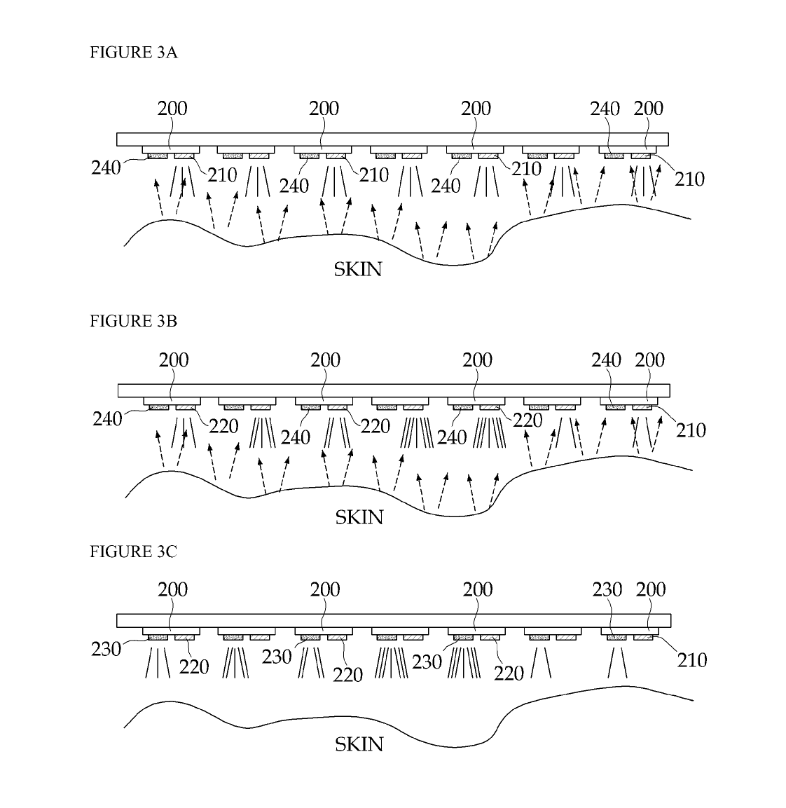

FIGS. 3A to 3C are exemplified diagrams for explaining the operational state of the phototherapy device, according to an exemplary embodiment of the present disclosure.

Referring to FIGS. 1 and 2, FIG. 3A shows the case where control unit 130 irradiates the first wavelength light to the skin through first light source 210 included in pixel 200 of pixel unit 150, and photodetector 240 detects the skin reflection light of the irradiated light. In this case, the wavelength of the first wavelength light irradiated to all the skin parts and the wavelength of the skin reflection light may be different from each other.

Control unit 130 controls the intensity of the second wavelength light for the skin diagnosis for each pixel 200 based on the first skin image information acquired through the skin reflection light of FIG. 3A, and performs a control to irradiate the second wavelength light having the controlled light intensity to the skin through second light source 220, as shown in FIG. 3B.

As shown in FIG. 3B, the intensity of light irradiated through second light source 220 may be the same or different for each pixel 200. The skin reflection light for the second wavelength light is detected by photodetector 240.

Control unit 130 derives the skin diagnosis results based on the second skin image information acquired through the skin reflection light for the second wavelength light, that is, the skin reflection light of FIG. 3B. Control unit 130 then determines the two-dimensional distribution of the third wavelength light for the skin disease therapy by using the derived results, and controls the intensity of the third wavelength light for each pixel 200 according to the two-dimensional distribution. Therefore, the intensity of light irradiated through third light source 230 is different for each pixel 200, similarly to FIG. 3B.

Control unit 130 performs a control to irradiate the third wavelength light having the controlled light intensity to the skin, through third light source 230, as shown in FIG. 3C.

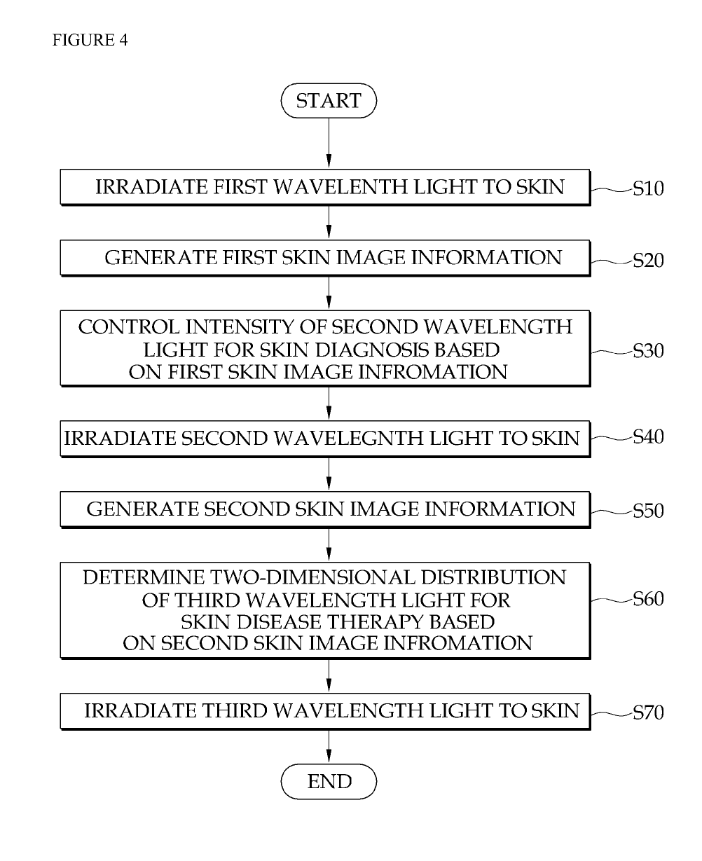

FIG. 4 shows a phototherapy method according to another exemplary embodiment of the present disclosure.

Referring to FIG. 4, the phototherapy device first irradiates the first wavelength light to the skin through first light source 210 (S10). The phototherapy device then generates the first skin image information from the first wavelength light irradiated and reflected to and from the skin (S20).

The phototherapy device controls the intensity of the second wavelength light for the skin diagnosis based on the first skin image information (S30). The phototherapy device irradiates the second wavelength light to the skin through second light source 220 using the controlled light intensity (S40).

The phototherapy device generates the second skin image information from the second wavelength light irradiated and reflected to and from the skin (S50). The phototherapy device determines the two-dimensional distribution of the third wavelength light for the skin disease therapy based on the second skin image information (S60).

The phototherapy device irradiates the third wavelength light to the skin through third light source 230 using the intensity of the third wavelength light according to the determined two-dimensional distribution of the light (S70).

As set forth above, when the phototherapy device according to the exemplary embodiment of the present disclosure is used, the skin diagnosis and therapy are simultaneously performed, such that the most suitable phototherapy may be performed according to the skin disease diagnosis.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A phototherapy device, comprising:
a pixel unit configured to irradiate light having a predetermined wavelength to a skin, detect a skin reflection light of the light irradiated to the skin, and convert the detected skin reflection light into an electrical signal;
an image processor configured to use the electrical signal to generate skin image information; and
a control unit configured to control light intensity irradiated to the skin, or to determine two-dimensional distribution of light for a skin diagnosis or a skin disease therapy based on the skin image information.

2. The device of claim 1, wherein the pixel unit includes at least one pixel and the pixel includes: a first light source to irradiate a first wavelength light in order to acquire skin image information, a second light source to irradiate a second wavelength light for a skin diagnosis, a third light source to irradiate a third wavelength light for a skin disease therapy, and a photodetector to detect the skin reflection light and convert the detected skin reflection light into the electrical signal.

3. The device of claim 2, wherein when the pixels are in plural, the plurality of pixels are arranged either in two-dimensionally or three-dimensionally.

4. The device of claim 2, wherein the control unit controls the intensity of the second wavelength light based on the first skin image information on the skin reflection light of the first wavelength light irradiated to the skin.

5. The device of claim 4, wherein the control unit controls the intensity of the second wavelength light for each pixel when the pixels of the pixel unit are in plural.

6. The device of claim 4, wherein the control unit derives skin diagnosis results based on second skin image information on the skin reflection light of the second wavelength light irradiated to the skin, and determines two-dimensional distribution of the third wavelength light for the skin disease therapy based on the skin diagnosis results.

7. The device of claim 6, wherein the control unit controls the intensity of the third wavelength light for each pixel when the pixels of the pixel unit are in plural.

8. The device of claim 1, wherein the controller analyzes information on a skin-related structure including a skin surface state, a thickness of a skin dermis, a density of a skin dermis, and a position and a shape of a skin disease from the skin image information.

9. The device of claim 1, further comprising a memory storing the skin image information and the skin diagnosis results.

10. The device of claim 1, wherein the wavelength of light irradiated to the skin by the pixel unit ranges from about 400 nm to about 900 nm.

11. A pixel module for phototherapy including pixels arranged in plural, the pixel module comprising:
a first light source to irradiate a first wavelength light in order to acquire skin image information;
a second light source to irradiate a second wavelength light for a skin diagnosis;
a third light source to irradiate a third wavelength light for a skin disease therapy; and
a photodetector to detect light irradiated and reflected to and from the skin from the first light source or the second light source, and convert the detected light into an electrical signal.

12. The pixel module of claim 11, wherein each of variables including an output density, a spot size, an operation time, a radiation repetition time, and a radiation repetition period of the light source is independently controlled for each pixel.

13. A phototherapy method, comprising:
irradiating a first wavelength light to a skin;
generating first skin image information from skin reflection light of the first wavelength light;
controlling intensity of a second wavelength light for a skin diagnosis based on the first skin image information;
irradiating the second wavelength light having the controlled light intensity to the skin;
generating second skin image information from the skin reflection light of the second wavelength light;
performing the skin diagnosis based on the second skin image information and determining a two-dimensional distribution of a third wavelength light for a skin disease therapy according to the skin diagnosis results; and
irradiating the third wavelength light having the two-dimensional distribution determined at the performing step to the skin.

14. The method of claim 13, further comprising storing the skin image information and the skin diagnosis results into a memory device.
